(19) United States
(12) Patent Application Publication
(10) Pub. No.: US 2024/0123181 A1
(43) Pub. Date: Apr. 18, 2024

(54) URINARY CATHETER WITH PATTERNED DRAINAGE HOLES TO PROVIDE TIP FLEXIBILITY

(71) Applicant: Hollister Incorporated, Libertyville, IL (US)

(72) Inventors: Satwinder S. Panesar, Foxford (IE); Heather O'Connor, Ballina (IE); Michael G. Murray, Ballina (IE); Paul Healy, Claremorris (IE)

(21) Appl. No.: 18/554,779

(22) PCT Filed: Apr. 14, 2022
(86) PCT No.: PCT/US2022/024778

§ 371 Date: Oct. 10, 2023

Related U.S. Application Data
(60) Provisional application No. 63/175,962, filed on Apr. 16, 2021.

(51) Int. Cl. A61M 25/00

(52) U.S. Cl. CPC A61M 25/0017; A61M 25/007; A61M 25/008; A61M 2025/0081; A61M 2210/1085

(57) ABSTRACT

Urinary catheters (100) that have drainage holes (112) that impart a desired flexibility to the proximal insertion end portion of the catheter.

The present application claims the benefit of and priority to U.S. Provisional Patent Application No. 63/175,962, filed Apr. 16, 2021, which is hereby incorporated herein by reference.

### FIELD OF THE DISCLOSURE

This disclosure relates generally to urinary catheters that include shafts having flexible proximal insertion end portions. More particularly, this disclosure relates to intermittent urinary catheters wherein the proximal insertion end portions of the catheter shafts include a plurality of drainage holes in a pattern that imparts selected flexibility to the proximal insertion end portion.

### BACKGROUND

Intermittent urinary catheters include a catheter shaft having a proximal insertion end portion that is inserted through the urethra and into the bladder. Once in the bladder, the urine enters the catheter through one or more drainage holes. The urine enters into the drainage hole(s) drains through a drainage lumen of the catheter and out of a distal drainage opening in the distal end portion of the catheter shaft.

FIG. 1A illustrates a prior art example of an intermittent urinary catheter. Catheter 10 includes an elongated catheter shaft 12 having a proximal insertion end portion 14 and a distal end portion 16. Proximal insertion end portion 14 includes a proximal end insertion tip 18 that is suitable for insertion into the urethra. Proximal end insertion tip 18 includes draining holes or eyelets 20 for receiving urine therethrough and into an internal conduit or lumen of catheter shaft 12. Distal end portion 16 may include a distal opening that is in fluid communication with a drainage member 22, such as a funnel, for fluidly connecting catheter 10 to a collection container, such as a collection bag, or for directing urine to a waste receptacle, such as a toilet.

In some instances, it is preferable or desired for a urinary catheter to have a flexible proximal insertion end portion. As shown in FIGS. 1B and 1C, some catheters provide flexible insertion tips by thinning out the neck region 24. Such catheters typically include an olive/spherical sphere tip 26. Such designs result in the eyelets 20 being positioned further back from the tip 26 in order to accommodate the longer necked region 24. Accordingly, such catheters are relatively longer, which results in a longer portion of the catheter being inserted into the bladder, and such relatively long catheters may require additional material to form the catheter.

There remains a need for catheters with improved flexible tips.

### SUMMARY

In one aspect, an intermittent urinary catheter includes a catheter shaft having a proximal insertion end portion for advancement through the urethra into a bladder and a distal drainage end portion having a drainage opening. The catheter shaft has a drainage lumen in communication with the drainage opening. The proximal insertion end portion of the catheter shaft includes a terminal proximal end and a drainage hole region distal of the terminal proximal end. The drainage hole region includes a plurality of drainage holes extending through the proximal insertion end portion of the catheter and in communication with the drainage lumen. The plurality of drainage holes are in a pattern that is configured so that the drainage hole region has a greater flexibility than a portion of the catheter shaft distal to the drainage hole region.

### BRIEF DESCRIPTION OF THE DRAWINGS

FIG. 1A is a side elevational view of a prior art urinary catheter;

FIG. 1B is a side elevational view of another prior art urinary catheter;

FIG. 1C is a side elevational view of another prior art urinary catheter;

FIG. 2 is a partial side elevational view of one embodiment of a urinary catheter in accordance with the present disclosure;

FIG. 3 is a cross-sectional view the of the proximal insertion end portion of the urinary catheter of FIG. 2;

FIG. 4 is side view of the proximal insertion end portion of the urinary catheter of FIG. 2, shown with a force being applied to the proximal insertion end portion;

FIG. 5 is a partial side elevational view of another embodiment of a urinary catheter in accordance with the present disclosure;

FIG. 6 is a partial top view the of the urinary catheter of FIG. 5;

FIG. 7 is perspective view of the proximal insertion end portion of the urinary catheter of FIG. 5;

FIG. 8 is a partial cross-sectional view the of the proximal end portion of the urinary catheter of FIG. 5;

FIG. 9 is side view of the proximal insertion end portion of the urinary catheter of FIG. 5, shown with a force being applied to the proximal insertion end portion;

FIG. 10 is a partial side elevational view of another embodiment of a urinary catheter in accordance with the present disclosure;

FIG. 11 is perspective view of the proximal insertion end portion of the urinary catheter of FIG. 10;

FIG. 12 is side view of the proximal insertion end portion of the urinary catheter of FIG. 10, shown with a force being applied to the proximal insertion end portion;

FIG. 13 is a partial side elevational view of another embodiment of a urinary catheter in accordance with the present disclosure;

FIG. 14 is perspective view of the proximal insertion end portion of the urinary catheter of FIG. 13;

FIG. 15 is side view of the proximal insertion end portion of the urinary catheter of FIG. 13, shown with a force being applied to the proximal insertion end portion;

FIG. 16 is a partial side elevational view of another embodiment of a urinary catheter in accordance with the present disclosure;

FIG. 17 is perspective view of the proximal insertion end portion of the urinary catheter of FIG. 16; and

FIG. 18 is side view of the proximal insertion end portion of the urinary catheter of FIG. 16, shown with a force being applied to the proximal insertion end portion.

### DETAILED DESCRIPTION OF THE EMBODIMENTS

The present disclosure is directed to intermittent urinary catheters having flexible proximal insertion tips, wherein the drainage holes of the urinary catheter are positioned relatively close to the proximal terminal end of the catheter shaft and impart a selected or desired flexibility to the proximal insertion tip of the catheter shaft.

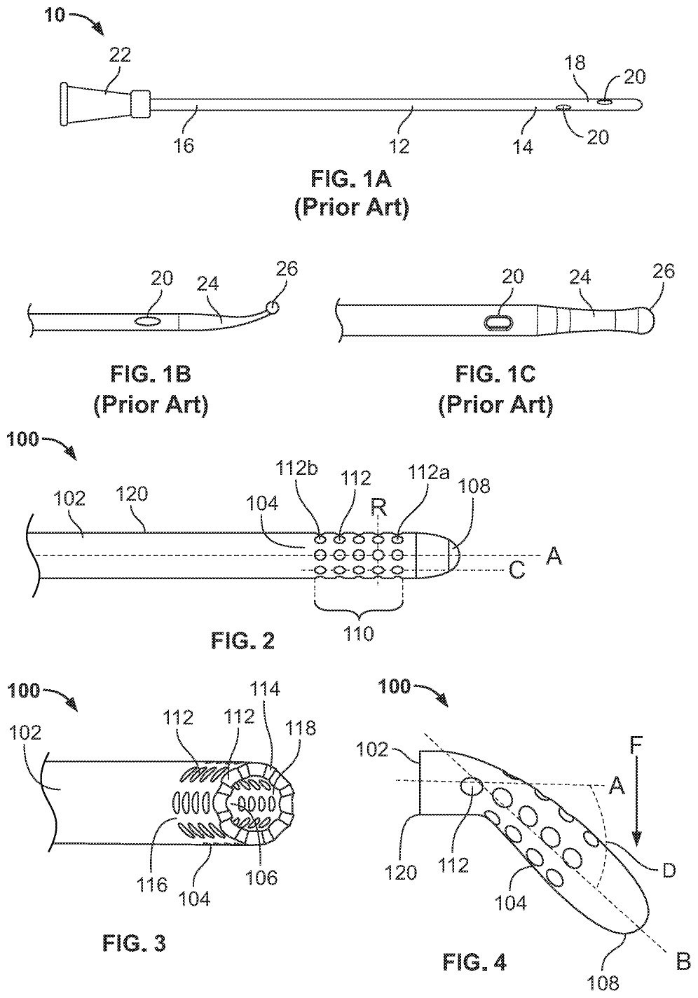

Turning now to FIGS. 2-4, there is shown one embodiment of a urinary catheter 100 of the present disclosure. The urinary catheter 100 includes a catheter shaft 102 having a proximal insertion end portion 104. Similar to FIG. 1A, the urinary catheter 100 also may include a distal end portion (not shown). In this embodiment and the other embodiments disclosed herein, the distal end portion includes a drainage opening and, optionally, may include a drainage member (not shown) or be connected to a collection bag (not shown). Furthermore, the urinary catheter 100 includes a drainage lumen 106 (FIG. 3) that is in communication with the distal end drainage opening of the catheter shaft.

The proximal insertion end portion 104 includes a terminal proximal end 108. In this embodiment and the other embodiments disclosed herein, the terminal proximal end 108 may be a closed end or an open end. When the terminal end 108 is an open end, the terminal end includes an opening in communication with drainage lumen 106. The proximal insertion end portion 104 also includes drainage hole region 110 (FIG. 2) located distally of the proximal terminal end 108. The drainage hole region 110 includes a plurality of drainage holes 112 extending through the proximal insertion end portion 104 and in communication with the drainage lumen 106 (FIG. 3). Referring to FIG. 3, the urinary catheter shaft 102 includes a sidewall 114 having an outer surface 116 and an inner surface 118. The drainage holes 112 extend through the sidewall 114 from the outer surface 116 to the inner surface 118. The outer surface 116 of this embodiment, and in the other embodiments, may have a hydrophilic coating disposed thereon. Turning back to FIG. 2, the drainage hole region 110 extends along the catheter shaft 102 from the proximal most drainage hole 112a to the distal most drainage hole 112b. Furthermore, in this and other embodiments, the drainage hole region may be located between about 2 mm and about 30 mm, or preferably between about 5 mm and about 15 mm from the proximal terminal end 108.

The drainage holes 112 of this embodiment and other embodiments disclosed herein may be formed in any suitable manner, including but not limited to, mechanical punching, thermal forming, ultrasonic cutting, laser cutting, etc. The drainage holes 112 in this embodiment and other embodiments disclosed herein are in a patterned that imparts a selected or desired flexibility to the proximal insertion end portion 104, which allows the catheter to easily navigate the urethra.

In the illustrated embodiment, the drainage holes 112 are arranged in a plurality of adjacent columns C and adjacent rows R. Each column C has an axis parallel to the longitudinal axis A of the catheter shaft 102 and each row R extends circumferentially about the longitudinal axis A of the catheter shaft 102. In the illustrated embodiment, the rows R and columns C are in a generally uniform pattern. In alternative embodiments, the rows R and columns C may be offset or in a variable pattern. The distance between adjacent rows and adjacent columns may be the same or may vary. Additionally, in the illustrated embodiment, each of the drainage holes 112 has generally the same size and shape. For instances, the drainage holes 112 have a generally round shape. However, in other embodiments, the drainage holes may have other shapes, such as oval, oblong, polygonal or irregular. In one embodiment, the drainage holes 112 may have an opening between about 0.4 mm and about 2 mm and preferably about 0.72 mm and about 1.5 mm in their widest dimension, which may be a diameter when the drainage hole is generally round. For example, the opening be about 1.2 mm wide. In alternative embodiments, the drainage holes 112 may have various sizes and shapes relative to one another.

Referring to FIG. 4, the drainage holes 112 are sized, shaped, and patterned to impart a selected or desired flexibility to the proximal insertion end portion 104. For example, the flexibility of the drainage hole region 110 may be between 10% and 70% more flexible, preferably 30% and 50% more flexible, than the flexibility of a portion 120 of the catheter shaft 102 distal of the drainage hole region, as measured by an end loaded cantilever deflection test. Alternatively, or in addition to, when a force F of about 1.1 N is applied to the terminal end 108 of the catheter shaft while a portion distal of the proximal insertion end 104 is held in a liner configuration, the proximal insertion end 104 bends such that the axis B of the proximal insertion end extends about 30 to 50 degrees D relative to the longitudinal axis A of the catheter shaft 102. In one alternative, when 0.7 to 1.1 N of force is applied to the terminal proximal end in a direction perpendicular to the longitudinal axis of the catheter shaft, the drainage hole region bends such that the longitudinal axis of the drainage hole region is about 30 to 50 degrees, or about 40 degrees relative to the longitudinal axis of the catheter shaft.

In use, proximal insertion end portion 104 is inserted into a urethra. As the proximal insertion end portion 104 is advanced through the urethra, the proximal insertion end portion bends or flexes to assist in such advancement. The proximal insertion end portion is advanced until the drainage holes 112 enter the bladder. Because the drainage holes 112 are relatively close to the proximal terminal end 108 of the catheter shaft 102, only a relatively short length of the catheter shaft 102 is required to be inserted into the bladder. The urine is received into the drainage holes 112, flows through the drainage lumen 106 and out of the drainage opening (not shown) in the distal end portion of the catheter shaft.

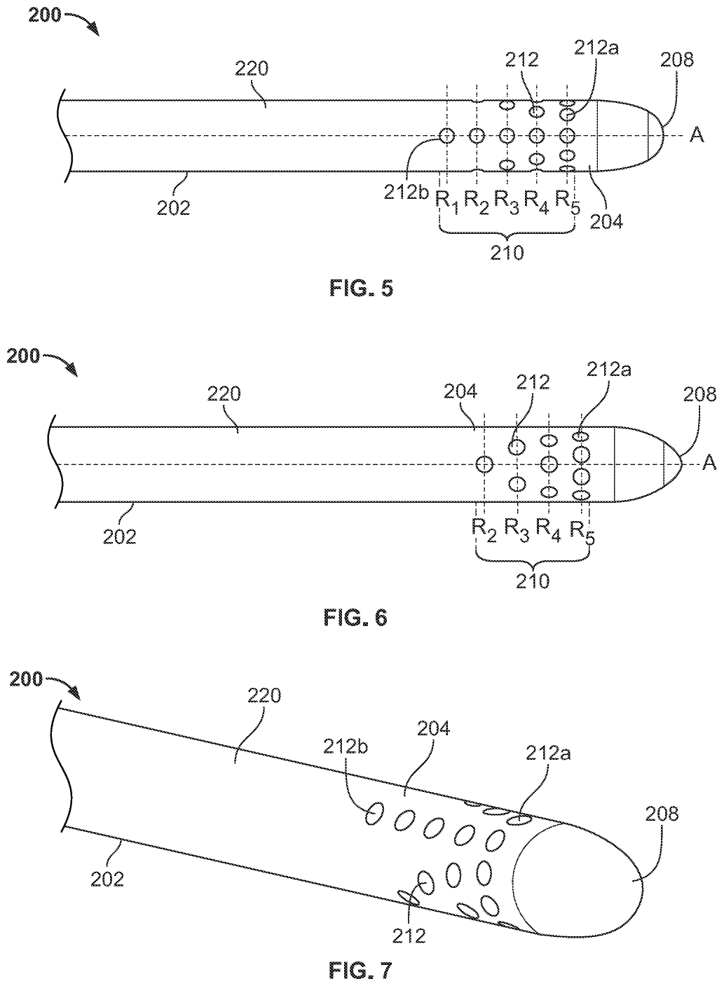

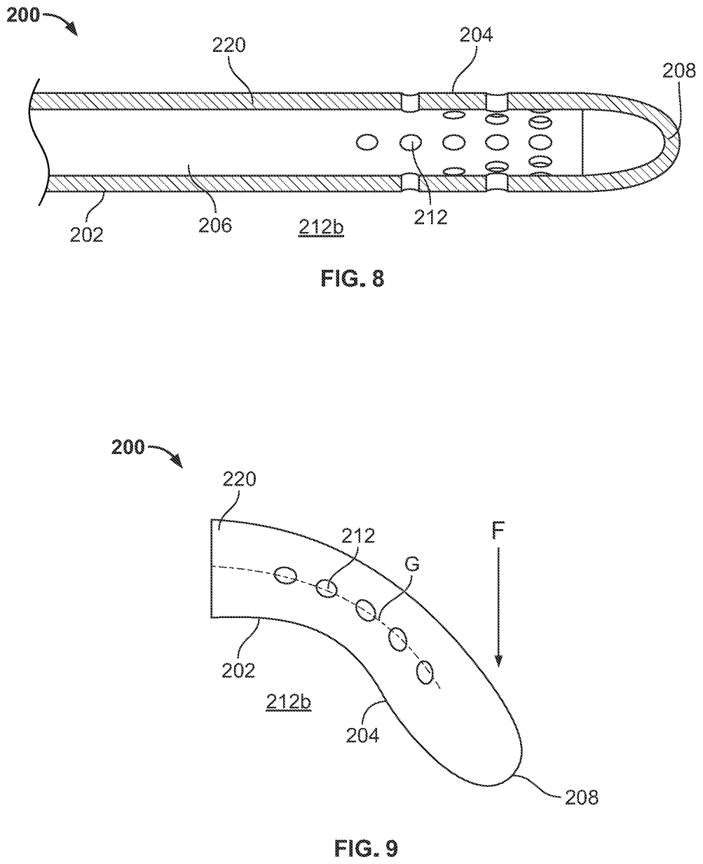

FIGS. 5-9 illustrate another embodiment of a urinary catheter 200 of the present disclosure. The urinary catheter 200 includes a catheter shaft 202 having a proximal insertion end portion 204. Furthermore, the urinary catheter 200 includes a drainage lumen 206 (FIG. 8) that is in communication with the distal end drainage opening (not shown) of the catheter shaft.

The proximal insertion end portion 204 includes a terminal proximal end 208, which may be a closed end or an open end, as described above. The proximal insertion end portion 204 also includes drainage hole region 210 (FIGS. 5 and 6) located distally of the proximal terminal end 208. The drainage hole region 210 includes a plurality of drainage holes 212 extending through the proximal insertion end portion 204 and in communication with the drainage lumen 206 (FIG. 8). The drainage hole region 210 extends along the catheter shaft 202 from the proximal most drainage hole 212a to the distal most drainage hole 212b.

The drainage holes 212 are in a patterned that imparts a selected or desired flexibility to the proximal insertion end portion 204. In the illustrated embodiment, the drainage holes 212 are in rows R that extend about the longitudinal axis A of the catheter shaft 202. At least some rows R₁-R₅ of drainage holes 212 have a lesser amount of drainage holes 212 than an immediately adjacent row of drainage holes. In the illustrated embodiment, the rows have less drainage holes 212 than immediate adjacent proximal rows. In other words, moving distally along the catheter shaft, at least some rows R₁-R₅ have a decreased number of drainage holes relative to proximal rows. In the illustrated embodiment, each row R₁-R₅ has less drainage holes than its immediately adjacent proximal row. In an alternative embodiment, at least some rows R₁-R₅ may have an increased number of drainage holes relative to adjacent distal rows. In the illustrated embodiment, the drainage holes 212 have a generally round shape. However, in other embodiments, the drainage holes may have other shapes, such as oval, oblong, or polygonal. The drainage holes 212 may have any of the sized described above. In alternative embodiments, the drainage holes 212 may have various sizes and shapes relative to one another.

Referring to FIG. 9, the drainage holes 212 are sized, shaped and pattern to impart a selected or desired flexibility to the proximal insertion end portion 204. In illustrated embodiment, the rows increasing in the amount of drainage holes toward the proximal terminal end 208 may result in variable flexibility along the proximal insertion end portion 204. For example, the proximal segment of the drainage hole region 210, that includes relatively more drainage holes, may have greater flexibility than the distal segment of the drainage hole region 210. In other words, drainage hole region 210 may transition in stiffness from a stiffer distal end segment to more flexible proximal one.

In one embodiment, flexibility of at least one segment of the drainage hole region 210 of the catheter shaft may be between 10% and 70% and preferably 30% and 50% more flexible than the flexibility of a portion 220 of the catheter shaft 202 distal of the drainage hole region, as measured by an end loaded cantilever deflection test. Alternatively, or in addition to, when a force F of about 1.1 N is applied to the terminal end 208 of the catheter shaft 202 while a portion distal of the proximal insertion end 204 is held in a liner configuration, the drainage hole region 210 of the proximal insertion end may bend in an arc or curvature G that decreases in radius of curvature from the distal end of the drainage hole region 210 to the proximal end of said region. In other words, the radius of curvature is smaller at the proximal end than at the distal end.

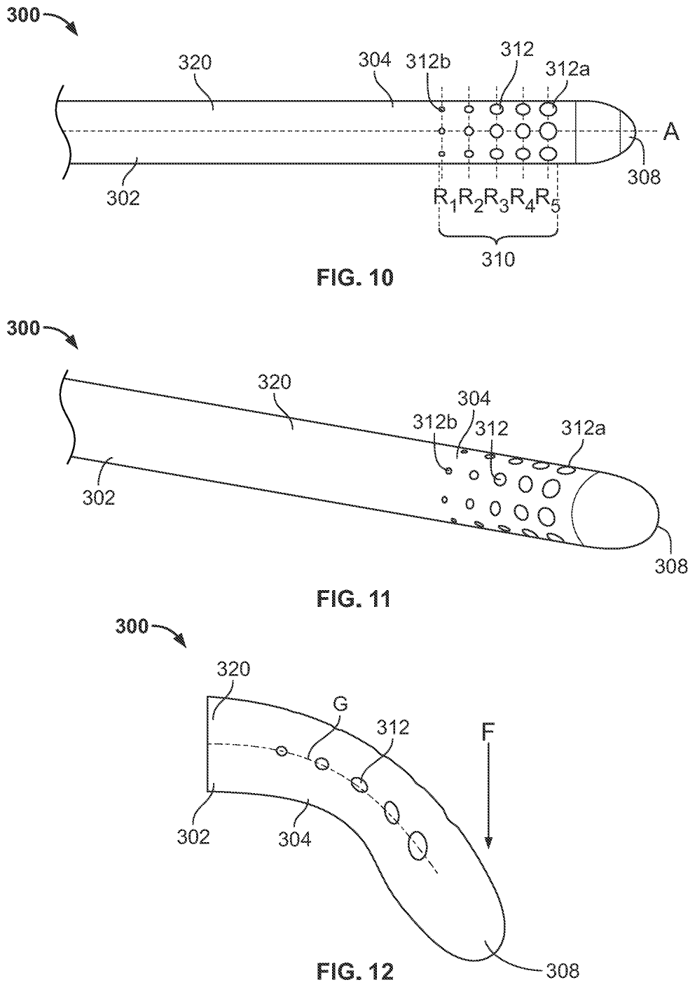

FIGS. 10-12 illustrate another embodiment of a urinary catheter 300 of the present disclosure. The urinary catheter 300 includes a catheter shaft 302 having a proximal insertion end portion 304. Furthermore, similar to the previous embodiments, the urinary catheter 300 includes a drainage lumen (not shown) that is in communication with the distal end drainage opening (not shown) of the catheter shaft.

The proximal insertion end portion 304 includes a terminal proximal end 308, which may be a closed end or an open end, as described above. The proximal insertion end portion 304 also includes drainage hole region 310 (FIG. 10) located distally of the proximal terminal end 308. The drainage hole region 310 includes a plurality of drainage holes 312 extending through the proximal insertion end portion 304 and in communication with the drainage lumen (not shown). The drainage hole region 310 extends along the catheter shaft 302 from the proximal most drainage hole 312a to the distal most drainage hole 312b.

The drainage holes 312 are in a patterned that imparts a selected or desired flexibility to the proximal insertion end portion 304. In the illustrated embodiment, the drainage holes 312 are in rows R, wherein at least some rows R₁-R₅ of drainage holes have smaller holes than proximal rows of drainage holes. In the illustrated embodiment, the rows have smaller drainage holes than immediate adjacent proximal rows. In other words, moving distally along the catheter shaft, at least some rows R₁-R₅ have drainage holes of decreased size relative to an adjacent row. In the illustrated embodiment, each row R₁-R₅ has drainage holes of a smaller size than the immediately adjacent proximal rows. In the illustrated embodiment, the drainage holes 312 have a generally round shape. However, in other embodiments, the drainage holes may have other shapes, such as oval, oblong, or polygonal. In one embodiment, the drainage holes 312 may have an opening between about 0.4 mm and about 2 mm, preferably about 0.73 mm and about 1.2 mm in their widest dimension, which may be a diameter when the drainage hole is generally round. Furthermore, the size of the holes in one row may be between about 10% and 70% smaller, or preferably 20% and 40% smaller, or preferably 20% than an adjacent row. Additionally, the size of the holes may decrease a constant rate (percentage or width) in a series of rows. The drainage holes 312 also may be arranged in uniform columns or in offset columns. In an alternative embodiment, the rows R₁-R₅ may have drainage holes of increased size relative to adjacent proximal rows.

Referring to FIG. 12, the drainage holes 312 are sized, shaped, and patterned to impart a selected or desired flexibility to the proximal insertion end portion 304. In illustrated embodiment, the increased size of drainage holes toward the proximal terminal end 308 may result in variable flexibility along the proximal insertion end portion 304. For example, the proximal segment of the drainage hole region 310, which includes relatively larger drainage holes, may have more flexibility than the distal segment of the drainage hole region. In one embodiment, flexibility of at least one segment of the drainage hole region 310 may be between 10% and 70%, preferably 30% and 50% more flexible than the flexibility of a portion 320 of the catheter shaft 302 distal of the drainage hole region, as measured by an end loaded cantilever deflection test. Alternatively, or in addition to, when a force F of about 1.1 N is applied to the terminal end 308 of the catheter shaft 302 while a portion distal of the proximal insertion end 304 is held in a liner configuration, the drainage hole region 310 of the proximal insertion end may bend in an arc or curvature G that decreases in radius of curvature from the distal end of the drainage hole region 310 to the proximal end of said region. In other words, the radius of curvature is smaller at the proximal end than at the distal end.

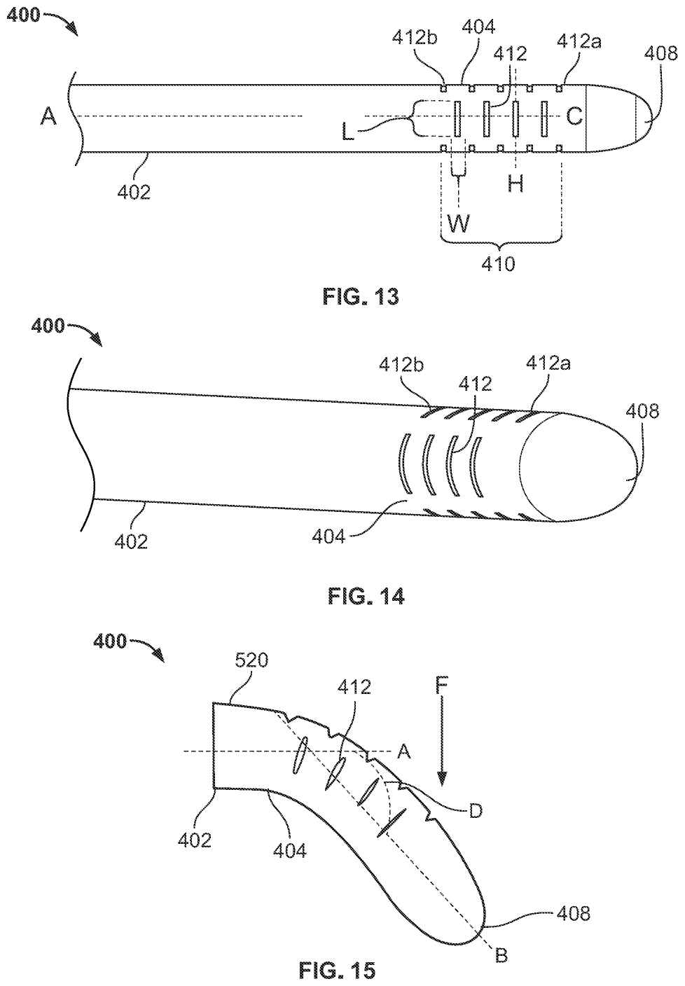

FIGS. 13-15 illustrate another embodiment of a urinary catheter 400 of the present disclosure. The urinary catheter 400 includes a catheter shaft 402 having a proximal insertion end portion 404. Similar to that described above, the urinary catheter 400 includes a drainage lumen 406 (now shown) that is in communication with the distal end drainage opening (not shown) of the catheter shaft.

The proximal insertion end portion 404 includes a terminal proximal end 408, which may be a closed end or an open end. The proximal insertion end portion 404 also includes drainage hole region 410 (FIG. 13) located distally of the proximal terminal end 408. The drainage hole region 410 includes a plurality of drainage holes 412 extending through the proximal insertion end portion 404 and in communication with the drainage lumen (not shown). The drainage hole region 410 extends along the catheter shaft 402 from the proximal most drainage hole 412a to the distal most drainage hole 412b.

The drainage holes 412 are in a patterned that imparts a selected or desired flexibility to the proximal insertion end portion 404. In the illustrated embodiment, the drainage holes 412 are elongated slots or slits having an elongated arc or axis H that extends partially along the outer circumference of the catheter shaft 402. The drainage holes 412 may have a length L of 2 mm to 3 mm, preferably 2.6 mm and a width W of 0.1 to 0.3 mm and preferably 0.2 mm. Additionally, the slits may be aligned in offset columns C. The columns C have an axis that is parallel with the axis A of the catheter shaft 402.

Referring to FIG. 15, the drainage holes 412 are sized, shaped, and patterned to impart a selected or desired flexibility to the proximal insertion end portion 404. For example, the flexibility of the drainage hole region 410 may be between 10% and 70% preferably 50% and 70% more flexible than the flexibility of a portion 420 of the catheter shaft 402 distal of the drainage hole region, as measured by an end loaded cantilever deflection test. Alternatively, or in addition to, when a force F of about 0.7 N is applied to the terminal end 408 of the catheter shaft while a portion distal of the proximal insertion end 404 is held in a liner configuration, the proximal insertion end 404 bends such that the axis B of the proximal insertion end extends about 40 to 60 degrees or preferably 50 degrees D relative to the longitudinal axis A of the catheter shaft 402.

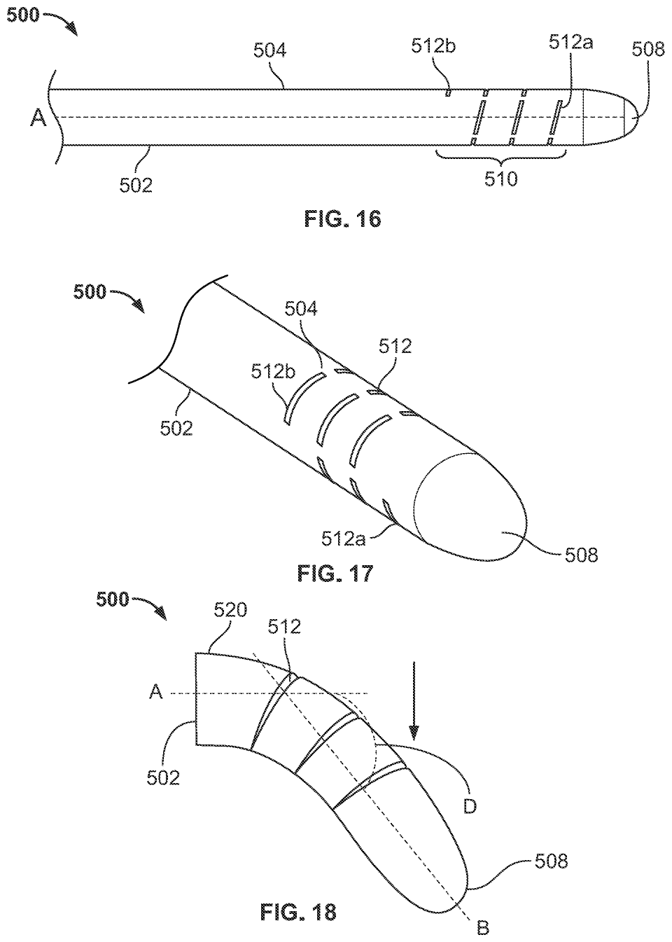

FIGS. 16-18 illustrate another embodiment of a urinary catheter 500 of the present disclosure. The urinary catheter 500 includes a catheter shaft 502 having a proximal insertion end portion 504. Similar to that described above, the urinary catheter 500 includes a drainage lumen 506 (not shown) that is in communication with the distal end drainage opening (not shown) of the catheter shaft.

The proximal insertion end portion 504 includes a terminal proximal end 508, which may be a closed end or an open end. The proximal insertion end portion 504 also includes drainage hole region 510 (FIG. 16) located distally of the proximal terminal end 508. The drainage hole region 510 includes a plurality of drainage holes 512 extending through the proximal insertion end portion 504 and in communication with the drainage lumen 506 (not shown). The drainage hole region 510 extends along the catheter shaft 502 from the proximal most drainage hole 512a to the distal most drainage hole 512b.

The drainage holes 512 are in a patterned that imparts a selected or desired flexibility to the proximal insertion end portion 504. In the illustrated embodiment, the drainage holes 512 are slots having a size and shape similar to that described above with respect to FIGS. 13-15. In this embodiment, the slots are patterned in a helical pattern or configuration about axis A of the catheter shaft 502.

Referring to FIG. 18, the drainage holes 512 are sized, shaped, and patterned to impart a selected or desired flexibility to the proximal insertion end portion 504. For example, the flexibility of the drainage hole region 510 may be between 30% and 70%, preferably 40% and 60% more flexible than the flexibility of a portion 520 of the catheter shaft 502 distal of the drainage hole region, as determined by an end loaded cantilever deflection test. Alternatively, or in addition to, when a force F of about 0.9 N is applied to the terminal end 508 of the catheter shaft while a portion distal of the proximal insertion end 504 is held in a liner configuration, the proximal insertion end 504 bends such that the axis B of the proximal insertion end extends about 40 to 60 degrees, preferably 50 degrees D relative to the longitudinal axis A of the catheter shaft 502.

It should be understood that various changes and modifications to the presently preferred embodiments described herein will be apparent to those skilled in the art. Such changes and modification can be made without departing from the spirit and scope of the invention disclosed herein.

What is claimed is:

1. A intermittent urinary catheter, comprising:
a catheter shaft having a proximal insertion end portion for advancement through a urethra into a bladder and a distal drainage end portion having a drainage opening, the catheter shaft having a drainage lumen in communication with the drainage opening;
the proximal insertion end portion comprising a terminal proximal end and a drainage hole region distal of the terminal proximal end, the drainage hole region including a plurality of drainage holes extending through the proximal insertion end portion of the catheter and in communication with the drainage lumen; and
wherein the plurality of drainage holes are in a pattern that is configured so that the drainage hole region has a greater flexibility than a portion of the catheter shaft distal to the drainage hole region.

2. The intermittent urinary catheter of claim 1, wherein the flexibility of the drainage hole region is 10% to 70% more flexible than a flexibility of the portion of the catheter shaft distal of the drainage hole region, as measured by an end loaded cantilever deflection test.

3. The intermittent urinary catheter of claim 1, wherein the catheter shaft includes a sidewall having an outer surface and an inner surface, wherein the inner surface defines the drainage lumen;
the plurality of drainage holes extend through the sidewall; and
the drainage hole region of the proximal insertion end portion extending from a most proximal drainage hole through the sidewall to a most proximal drainage hole through the sidewall.

4. The intermittent urinary catheter of claim 1, wherein the drainage hole region varies in flexibility along a longitudinal axis of the catheter shaft.

5. The intermittent urinary catheter of claim 4, wherein the proximal end of the drainage hole region has a greater flexibility than the distal end of the drainage hole region.

6. The intermittent urinary catheter of claim 1, wherein the drainage hole region has a longitudinal axis; and
when 0.7 to 1.1 N of force is applied to the terminal proximal end in a direction perpendicular to the longitudinal axis of the catheter shaft, the drainage hole region bends such that the longitudinal axis of the drainage hole region is about 30 to 50 degrees relative to the longitudinal axis of the catheter shaft.

7. The intermittent urinary catheter of claim 1, wherein the drainage hole region is located between 2 mm and 30 mm.

8. The intermittent urinary catheter of claim 1, wherein the pattern of the plurality of drainage holes comprises the drainage holes being arranged in a plurality of columns and a plurality of rows, each column having an axis parallel to the longitudinal axis of the catheter shaft and each row extending circumferentially about the longitudinal axis of the catheter shaft.

9. The intermittent urinary catheter of claim 8, wherein the each of the drainage holes have the same shape and same size.

10. The intermittent urinary catheter of claim 9, wherein at least some rows of drainage holes have a smaller size than an immediately adjacent row of drainage holes.

11. The intermittent urinary catheter of claim 9, wherein each row of drainage holes has a smaller size than an immediately adjacent proximal row of drainage holes.

12. The intermittent urinary catheter of claim 9, wherein each row of drainage holes has a smaller size than an immediately adjacent distal row of drainage holes.

13. The intermittent urinary catheter of claim 8, wherein each row of drainage holes is the same distance from adjacent rows.

14. The intermittent urinary catheter of claim 8, wherein a distance between adjacent rows varies.

15. The intermittent urinary catheter of claim 8, wherein each column of drainage holes is the same distance from adjacent columns.

16. The intermittent urinary catheter of claim 8, wherein a distance between adjacent columns varies.

17. The intermittent urinary catheter of claim 8, wherein the rows are offset from adjacent rows.

18. The intermittent urinary catheter of claim 17, wherein each row is offset from an immediately adjacent row.

19. The intermittent urinary catheter of claim 17, wherein the drainage holes comprise elongated slots.

20. The intermittent urinary catheter of claim 19, wherein the elongated slots are elongated.

21. The intermittent urinary catheter of claim 1, wherein the pattern of the plurality of drainage holes comprises a plurality of adjacent rows wherein rows have less drainage holes than an adjacent proximal rows.

22. The intermittent urinary catheter of claim 21, wherein each row has less drainage holes than an immediately adjacent proximal rows.

23. The intermittent urinary catheter of claim 1, the pattern of the plurality of drainage holes comprises elongated slots extending in a helical pattern about the catheter shaft.
